(19) United States
(12) Patent Application Publication
(10) Pub. No.: US 2018/0169293 A1
(43) Pub. Date: Jun. 21, 2018

(54) COLLAGEN DOUGH AND METHOD FOR PRODUCING COLLAGEN DOUGH CONTAINING CHITOSAN

(71) Applicant: Nitta Casings Inc., Bridgewater, NJ (US)

(72) Inventors: Eugene Goldfarb, Marlboro, NJ (US); Masayuki Mimasu, Bound Brook, NJ (US); Rod Moore, Bedminster, NJ (US); Richard E. Battersby, Frenchtown, NJ (US)

(21) Appl. No.: 15/843,003

(22) Filed: Dec. 15, 2017

Related U.S. Application Data
(60) Provisional application No. 62/468,196, filed on Mar. 7, 2017.
(60) Provisional application No. 62/434,550, filed on Dec. 15, 2016.

(51) Int. Cl. A61L 15/32; A61L 15/20; A61L 15/18; A61L 15/28; A61L 15/44; A61K 31/722

(52) U.S. Cl. CPC A61L 15/325; A61L 15/20; A61L 15/18; A61L 15/28; A61L 2300/21; A61K 31/722; A61L 2300/404; A61L 2300/106; A61L 15/44

(57) ABSTRACT

A hydrolyzed collagen dough produced from bovine collagen and methods for producing the collagen dough from bovine hides is provided. The collagen dough comprises water, bovine collagen, sodium benzoate and hydrochloric acid. To produce the collagen dough, the corium layer is separated from bovine hides and treated. The collagen dough may be used, for example, in biomedical applications, such as cell technology/tissue engineering, suture materials, or film and sponges for skin grafting. The collagen dough may further comprise chitosan.

This application claims priority under 35 U.S.C. § 119(e) to U.S. Provisional Patent Application No. 62/434,500 titled “Collagen Dough and Method for Producing Collagen Dough” filed on Dec. 15, 2016, and U.S. Provisional Patent Application No. 62/468,196 titled “Collagen Dough and Method for Producing Collagen Dough Containing Chitosan”, filed on Mar. 7, 2017, the entire contents of each of which are hereby incorporated by reference.

### FIELD OF THE INVENTION

The present invention is generally directed to a collagen dough and methods for producing the collagen dough. The collagen dough is produced using reconstituted bovine collagen and other food grade ingredients. The collagen dough can be used, for example, in medical applications to produce dressings for burns or other wounds. In one embodiment, the collagen dough contains chitosan.

### BACKGROUND

Collagen is a natural component of skin, and as such, is extremely compatible with the human body. Hydrolyzed collagen give a protective coating effect against irritation from exposure to surface active agents or other irritants. The result is known as the “protective colloid effect” and is the basis for the use of collagen in cosmetics.

Bovine collagen has been used for many years to produce products such as casings for sausages, frankfurters and other meat products. The collagen is cut, treated to form a gel-like material, extruded to form casings, dried and shirred.

The present invention is directed to a novel use of bovine collagen to form a collagen dough that can be used to produce various products, including medical products such as dressings for wounds or burns. The collagen dough may further comprise chitosan. Chitosan is hypoallergenic and has natural antibacterial properties. In addition, chitosan has properties allowing it to rapidly clot blood. Accordingly, the inclusion of chitosan in collagen dough used for medical purposes may provide additional benefits.

The invention is not limited in this regard and the collagen dough may be used in any appropriate application.

### SUMMARY OF THE INVENTION

The present invention is directed to a collagen dough and methods of making the collagen dough. The collagen dough is comprised of hydrolyzed bovine collagen.

The collagen dough is made using the corium layer of cattle hides. The corium layer of cattle hides are received in a limed form and stored in a refrigerated storage room. “Hides” is used herein to refer to the corium collagen layer that is used to produce the collagen dough. The hides are washed in drums in a 24-hour batch process with ammonium sulfate solution to remove the lime. The hides are then treated in a citric acid and sodium citrate buffer solution to adjust the pH of the hides. Following treatment in the buffer solution, the hides are washed and placed in cold storage until needed to produce collagen dough.

As needed, the washed hides are removed from storage and shredded and ground to a desired size. In one embodiment, the hides are ground to produce a quarter grind material, i.e. ground hides having a particle size of about one quarter inch.

The ground hides are transferred to a tank where they are mixed with water and agitated. The ground hide and water suspension undergoes a micro-cutting process to produce a pulp material. A mixture of water, hydrochloric acid and sodium benzoate is then added to the pulp, and blended to form a gel. The gel is homogenized. From the homogenization state the gel is pumped into a polyethylene lined container. After packaging the collagen dough containers are stored in a refrigerated room.

In one embodiment, the collagen dough further comprises chitosan. Chitosan has natural anti-microbial properties making it desirable in a collagen dough used for medical purposes. In addition, the chitosan can extend the life of the collagen dough whether used for medical or other purposes.

Chitosan may be added to the collagen pulp with the sodium benzoate and hydrochloric acid in the mixing tank.

The process produces a collagen dough that can be further processed to produce various products, such as medical dressings for wounds or burns. The collagen dough, with or without added chitosan, may be processed to provide the material in any desired form for wound care. In one embodiment, the collagen dough may processed to form a powder. The powder may be applied to a wound directly, or it may be used to coat or impregnate a dressing made from another material such as a gauze. In another embodiment, the collagen dough may be rolled to any desired thickness and cut in any desired shape to form a collagen pad that may be applied to a wound. In another embodiment, the collagen dough may be mixed with carrier such as a liquid or incorporated in a gel to be applied to a wound. Any appropriate liquid or gel may be used.

Other objects and advantages of the present invention will become apparent to one skilled in the art in view of the Description of the Invention provided below.

### DESCRIPTION OF THE FIGURES

FIG. 1 is a schematic showing one embodiment of a process for producing collagen dough from bovine collagen.

### DESCRIPTION OF THE INVENTION

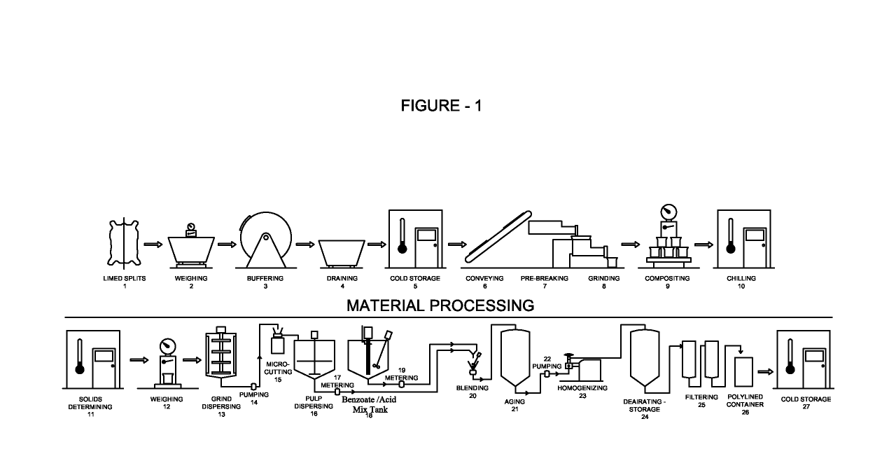

Collagen dough is manufactured by treating bovine collagen to produce a gel-like collagen product. The collagen dough may be used to produce medical products such as dressings for wounds or burns. One embodiment of a process for producing collagen dough is shown in FIG. 1 and described below. It should be understood that the description provided below is not meant to limit the scope of the invention, and one skilled in the art will readily understand that process parameters such as volumes, temperatures, times and concentrations may be varied.

In one embodiment of the invention, the corium layer of cattle hides are received in a limed form and stored in a refrigerated storage room. “Hides” is used herein to refer to the corium collagen layer that is used to produce the collagen dough. Typically, the hides are maintained at a temperature of 0-4° C. until further processed as described below.

The hides are first processed to prepare the corium material for conversion into collagen dough. The hides are weighed 2 and treated in a buffering drum 3. The buffering drum may be sized to handle, for example, up to 3000 Kg of product. In the buffering drum, the hides are treated with an ammonium sulfate solution to remove the lime, then water washed. In one embodiment, the concentration of the ammonium sulfate solution is in the range of about 0.5%-2.2% (w/w). The hides may be treated for up to about 24 hours with ammonium sulfate.

The hides are then further treated with a citric acid/sodium citrate buffer solution, followed by another water wash. In one embodiment, the citric acid/sodium citrate buffer solution has a concentration of about 0.0725-0.29% (w/w). The buffering process brings the pH of the corium layer down to approximately 4.3 to 4.9. Assays are run to ensure the product reaches the appropriate pH. The buffered hides are drained 4 and then are placed in cold storage 5 at a temperature of between about 0-4° C. until needed for further processing.

When additional material is needed for processing to produce the collagen dough, the buffered hides are sent on a conveyor 6 to be shredded 7 and ground 8 to a desired size for further treatment. In one embodiment, the hides are shredded and ground to produce a quarter grind (i.e., a material having a particle size of about one-quarter inch) while being maintained at a temperature of less than approximately 25° C. The invention is not limited in this regard, and the hides may be shredded to any desired size. The ground material is composited 9 and chilled 10 and maintained at a temperature of about 18° C.

The chilled ground material is weighed 12 and transferred to a mixing tank 13 where it is mixed with water and agitated. The ground material/water suspension undergoes a micro-cutting process 15 to further reduce the particle size of the ground hides and produce a corium pulp material. The corium pulp material is mixed in a dispersing tank 16. A mixture of water, hydrochloric acid and sodium benzoate is mixed in tank 18 is blended with the pulp 20 and stored in an aging tank 21 where the collagen and chemicals react to form a gel-like collagen dough material. In one embodiment, the amount of hydrochloric acid and sodium benzoate in tank 18 is controlled to achieve a concentration of about 0.25 to 2.0 wt % sodium benzoate and between about 0.16 to 2.0 wt % hydrochloric acid in the aging tank 21.

The collagen and chemicals are typically blended and maintained in the aging tank 21 at a temperature of between about 14° C. to 23° C. for a period of between about 12 to 24 hours. The collagen dough product so obtained has the following approximate composition:

| Ingredient | Percent by Weight |
|---|---|
| Hide Solids (i.e. bovine collagen) | About 4 to 10 wt % |
| Sodium Benzoate | About 0.25 to 2.0 wt % |
| Hydrochloric Acid | About 0.16 to 2.0 wt % |
| Water | Balance |

In some embodiments, the collagen dough comprises between about 5 percent and about 8 percent by weight bovine collagen, between about 0.5 percent and about 1.5 percent by weight sodium benzoate, and between about 0.5 percent to about 1.5 percent by weight hydrochloric acid. In one embodiment, the collagen dough comprises about 5.08 wt % collagen, about 0.182 wt % hydrochloric acid and about 0.102 wt % sodium benzoate.

The collagen dough is homogenized in tank 23 and pumped into a polyethylene lined container 24. The collagen dough is pumped through filters 25 and packaged into poly lined containers 26. The collagen dough may be stored in a refrigerated room 27 maintained at about 2° C. to 5° C. At this temperature, the collagen dough typically has a shelf life of at least one year.

The collagen dough produced by the process described above typically has a clear or white appearance. The pH of the collagen dough is typically about 2.0-3.5. The viscosity of the collagen dough is typically greater than about 1,000,000 centiPoise.

In another embodiment of the invention, the collagen dough further comprises chitosan. Chitosan is produced by deacetylation of chitin, which is the structural element in the exoskeleton of crustaceans such as crabs and shrimp. Chitosan has natural anti-microbial properties and can be used to replace other anti-microbial additives such as silver. In addition, chitosan can promote blood clotting, which makes it desirable for use in bandages or other wound covers.

In one embodiment, the chitosan added to the collagen dough has a degree of deacetylation of between 50% and 100% (w/v). The chitosan may have a degree of deacetylation of 89%. The chitosan may have a molecular weight of between about 300,000 and about 400,000 Daltons.

The chitosan may be added to the collagen pulp with the hydrochloric acid and sodium benzoate in the mix tank 18. In some embodiments, between about 0.75 wt % and about 2.75 wt % chitosan is added to the pulp material in the mixing tank. The amount of chitosan added may be between about 1.0 wt % and about 2.0 wt %. In one embodiment, the collagen dough comprises about 5.08 wt % collagen, about 0.3 wt % hydrochloric acid, about 0.102 wt % sodium benzoate and about 0.75% wt chitosan.

Due to its film forming and moisture binding properties, the collagen dough can improve the moisture content of the skin. The hydrolyzed collagen dough can potentiate epidermal metabolism by providing a suitable, moist environment on the surface of the epidermis for healthy skin and hair. In biomedical applications, the collagen dough can be used in cell technology/tissue engineering, suture materials, or film and sponges for skin grafting.

The collagen dough, with or without added chitosan, may be further processed to provide the material in any desired form for wound care. In one embodiment, the collagen dough may processed to form a powder. The powder may be applied to a wound directly, or it may be used to impregnate a dressing made from another material such as a gauze. In another embodiment, the collagen dough may be rolled to any desired thickness and cut in any desired shape to form a collagen pad that may be applied to a wound. In one embodiment, the thickness of the collagen pad is less than ¼ inch. In another embodiment, the collagen dough may be mixed with carrier such as a liquid or incorporated in a gel to be applied to a wound. Any appropriate liquid or gel may be used. In one embodiment, the liquid is water.

As will be recognized by those of ordinary skill in the pertinent art based on the teachings herein, numerous changes and modifications may be made to the above-described and other embodiments of the invention without departing from its scope. For example, the time, temperatures, pH, equipment size and other parameters described in the embodiments discussed above may be varied without departing from the scope of the invention. Accordingly, this detailed description is to be taken in an illustrative and exemplary as opposed to a limiting sense.

We claim:

1. A collagen dough comprising water, bovine collagen, sodium benzoate, and hydrochloric acid.

2. The collagen dough of claim 1, wherein the collagen dough comprises between about 4 percent and about 10 percent by weight bovine collagen, between about 0.25 percent and about 2 percent by weight sodium benzoate, and between about 0.16 percent to about 2 percent by weight hydrochloric acid.

3. The collagen dough of claim 1, wherein the collagen dough comprises between about 5 percent and about 8 percent by weight bovine collagen, between about 0.5 percent and about 1.5 percent by weight sodium benzoate, and between about 0.5 percent to about 1.5 percent by weight hydrochloric acid.

4. The collagen dough of claim 1, further comprising chitosan.

5. The collagen dough of claim 2, further comprising between about 0.75 wt % and about 2.75 wt % chitosan.

6. The collagen dough of claim 5, wherein the chitosan is between about 1.0 wt % and 2.0 wt %.

7. A process for producing a collagen dough comprising the steps of:
(a) treating the corium layer of limed bovine hides in an ammonium sulfate solution;
(b) treating the corium layer in a buffer solution comprising citric acid and sodium citrate;
(c) washing the treated corium layer with water;
(d) grinding the corium layer;
(e) mixing the ground corium with water and micro-cutting the corium to produce a corium pulp; and
(f) mixing the corium pulp with a solution comprising water, hydrochloric acid and sodium benzoate.

8. The process of claim 7, wherein the ammonium sulfate solution has a concentration of between 0.5% to 2.2%.

9. The process of claim 8, wherein the buffer solution has a concentration of between 0.0725% to 0.29%.

10. The process of claim 9, wherein following treatment in the buffer solution the hides have a pH of between about 4.3 to 4.9.

11. The process of claim 10, wherein the ground corium layer has a particle size of about one quarter of an inch.

12. The process of claim 11, wherein the corium pulp water, hydrochloric acid and sodium benzoate are maintained at a temperature of between about 14° C. to 23° C.

13. The process of claim 7, wherein between about 4 percent and about 10 percent by weight of the corium pulp is mixed with between about 0.25 percent and about 2 percent by weight sodium benzoate, and between about 0.16 percent to about 2 percent by weight hydrochloric acid.

14. The process of claim 7, wherein the solution mixed with the corium pulp further comprises chitosan.

15. The process of claim 13, wherein the corium pulp is further mixed with between about 0.75 wt % and about 2.75 wt % chitosan.

16. A material for wound care comprising collagen produced by the process of claim 15, wherein the collagen is a powder.

17. A material for wound care comprising collagen produced by the process of claim 15, wherein the collagen is in the form of a pad having a thickness of 1/4 inch or less.

18. A material for wound care comprising collagen produced by the process of claim 15, wherein the collagen is mixed with one of a liquid or a gel.

19. The material for wound care of claim 18, wherein the collagen is mixed with water.
